(19) United States
(12) Patent Application Publication
(10) Pub. No.: US 2017/0340410 A1
(43) Pub. Date: Nov. 30, 2017

(54) Cleaning System, Cleaning Devices, Instruction Insert, and Methods Therefor

(71) Applicant: Medline Industries, Inc., Mundelein, IL (US)

(72) Inventors: Sharbel Maalouf, Pleasant Prairie, WI (US); Mary Patricia Eble, Bellevue, WA (US)

(21) Appl. No.: 15/188,656

(22) Filed: Jun. 21, 2016

Related U.S. Application Data
(60) Provisional application No. 62/342,618, filed on May 27, 2016.

(51) Int. Cl. A61B 90/80; B08B 1/00; B65D 83/08; A61M 25/00

(52) U.S. Cl. CPC A61B 90/80; B65D 83/0805; B08B 1/006; A61M 25/0017

(57) ABSTRACT

A medical assembly (100) includes a package (101) having an outer surface (102) with a major face (103) defining an aperture (701). An aperture-sealing label (702) selectively attaches to the outer surface to close the aperture and to be selectively peelable from the outer surface to expose the aperture. Five sheets (302,303,304,305,306) are arranged in a stacked configuration (309) within the package. Printed instructions (107) then define five steps (110,111,112,113,114) instructing when to use each sheet disposed within the package, and when to discard the each sheet disposed within the package, to clean a Foley catheter insertion site of either a male patient or a female patient, while reducing chances for causing a catheter associated urinary tract infection.

### CROSS REFERENCE TO PRIOR APPLICATIONS

This application claims priority and benefit under 35 U.S.C. §119(e) from U.S. Provisional Application No. 62/342,618, filed May 27, 2016, which is incorporated by reference for all purposes.

### BACKGROUND

### Technical Field

This disclosure relates generally to cleaning systems, and more particularly to cleaning systems in accordance with predefined methods.

### Background Art

Healthcare facilities are increasingly concerned about the occurrence of secondary complications occurring during medical and surgical procedures. For example, during a medical procedure on an otherwise healthy patient, such as the insertion of a Foley catheter, there is the possibility that a secondary infection or other complication can result. This problem is so significant, it has been named with the acronym “CAUTI,” which stands for Catheter Associated Urinary Tract Infection. CAUTI primarily occurs at one or more of three events: Catheter insertion, maintenance of the catheter once inserted, and maintenance of the connected drain tubing and collection bag. It would be advantageous to have improved medical kits and associated methods and systems that help to prevent CAUTI.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present disclosure.

FIG. 1 illustrates one explanatory package assembly and a front side of one explanatory instruction insert in accordance with one or more embodiments of the disclosure.

FIG. 2 illustrates a second side of one explanatory instruction insert in accordance with one or more embodiments of the disclosure.

FIG. 3 illustrates a sectional view of one explanatory package assembly in accordance with one or more embodiments of the disclosure.

FIG. 4 illustrates one explanatory instruction insert, and one or more method steps, in accordance with one or more embodiments of the disclosure.

FIG. 5 illustrates another explanatory instruction insert, and one or more method steps, in accordance with one or more embodiments of the disclosure.

FIG. 6 illustrates another explanatory package assembly, shown with both front and rear sides, in accordance with one or more embodiments of the disclosure.

FIG. 7 illustrates one explanatory assembly in accordance with one or more embodiments of the disclosure.

FIG. 8 illustrates one explanatory method in accordance with one or more embodiments of the disclosure.

FIG. 9 illustrates another explanatory method in accordance with one or more embodiments of the disclosure.

FIG. 10 illustrates one or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. The terms “about” or “substantially” refer to items inclusive of manufacturing tolerances. Accordingly, a length of “about ten inches,” where the manufacturing tolerances were plus or minus two tenths of an inch, would include the range 9.8 to 10.2 inches, inclusive. Also, reference designators shown herein in parenthesis indicate components shown in a figure other than the one in discussion. For example, talking about a device (10) while discussing figure A would refer to an element, 10, shown in figure other than figure A.

A Foley catheter is a catheter inserted into the urinary tract through which a patient eliminates liquid waste. For example, a bedridden hospital patient may be catheterized with a Foley catheter so that they do not have to get up to urinate. Catheter insertion procedures are generally performed bedside or in a diagnostic lab room by a medical professional who specializes in catheter insertion. The medical professional is frequently a specially trained nurse.

Regardless of who inserts the Foley catheter, urinary tract infection is continually a concern. It will be readily understood that insertion of a foreign object, which can be on a semi-permanent basis, into a patient's urinary tract has associated therewith a risk that bacteria or other microbes will be introduced into the urinary tract during insertion. Studies have shown that such infections can be severe, and even a source of death. While the largest percentage of these infections occurs at the time of catheter insertion, significant amounts of infection can occur when the insertion site is being cleaned and maintained.

Embodiments of the present disclosure work to reduce the risk of urinary tract infections during catheter maintenance by providing an intuitive medical kit that assists medical personnel in executing method steps to clean Foley catheter insertion sites and otherwise maintain a Foley catheter installation while adhering to proper aseptic techniques that minimize the likelihood of infection. Embodiments of the disclosure include indicia configured as medical educational and instructional prompts that guide medical personnel through the steps of cleaning a Foley catheter insertion dressing while minimizing infection risk.

In one or more embodiments, a medical kit includes a predefined number of non-dispersible sheets. The non-dispersible sheets are in contrast to disposable wipes, which are designed to breakdown and disperse after use. The non-dispersible property associated with embodiments of the disclosure allows the sheets to be pre-moistened with rinse-free fluids that clean, moisturize, condition and soothe the skin. These fluids greatly aid in patient cleaning.

In one embodiment, a sheet package assembly includes a predefined number of sheets that corresponds to a specific Foley catheter site cleaning protocol. The sheet package assembly can be accompanied by instructions that provide a step-by-step method for cleaning a Foley catheter insertion site for a male or female patient. In one embodiment, this step-by-step method includes explicit instructions to discard, but not flush, the sheets at certain steps of the cleaning process.

For example, in one embodiment a method of cleaning a Foley catheter insertion site on a female comprises five steps. The instructions for executing this method on the medical educational and instructional prompts thus include five instructions to discard a cloth. Similarly, in one embodiment the method for cleaning a Foley catheter insertion site on a male includes five steps. The instructions for executing this method on the medical educational and instructional prompts would correspondingly include five instructions to discard a cloth. Accordingly, the health care services provider is apprised of the exact moment when each sheet should be discarded to help reduce the chance of the patient contracting a urinary tract infection.

In one embodiment, the predefined number of sheets is arranged in a stacked configuration. The stack of sheets is placed within a package. In one embodiment, the package defines an outer surface having a major face defining an aperture through which individual sheets can be drawn. An aperture-sealing label can then be attached to the package to conceal the aperture. In one embodiment, the aperture-sealing label is to selectively attach to the outer surface to close the aperture. In one embodiment, the aperture-sealing label is also selectively peelable from the outer surface to expose the aperture.

In one or more embodiments, the package includes medical indicia affixed thereto comprising educational prompts that instruct medical personnel regarding how to use each sheet disposed within the package, and expressly when to discard the same. For example, the medical indicia can feature animations and/or instructions that teach medical personnel how, when, and in what order to use each sheet, as well as when and in what order to discard each sheet after use. As the sheets are disposed within the package in a stacked configuration, they are advantageously placed in the order of use, with each sheet corresponding to a particular step of the method set forth in the medical indicia and/or educational prompts. This secondarily functions in the prevention of using the medical devices in an improper order.

Advantageously, embodiments of the disclosure overcome problems associated with prior art central catheter dressing replacement kits. Prior art central line dressing replacement kits are not arranged in a logical fashion so as to reduce the chance of CLABSI. Moreover, they fail to include the educational prompts and other indicia advantageously offered by embodiments of the present disclosure. As such, they are non-intuitive to use and require specialized training that few medical personnel possess. These deficiencies result in variation of procedure that can result in improper dressing change procedures that increase the risk of CLABSI.

Advantages offered by the embodiments of the disclosure, as compared to prior art kits, include helping medical personnel more easily clean the Foley catheter insertion site, while minimizing the chance for a urinary tract infection. Moreover, medical kits configured in accordance with one or more embodiments of the disclosure help to ensure medical personnel conformance with proper aseptic techniques. They also help in cleaning insertion sites without compromising the integrity of the catheter that has been inserted into the patient. Other advantages and benefits will be obvious to those of ordinary skill in the art having the benefit of this disclosure.

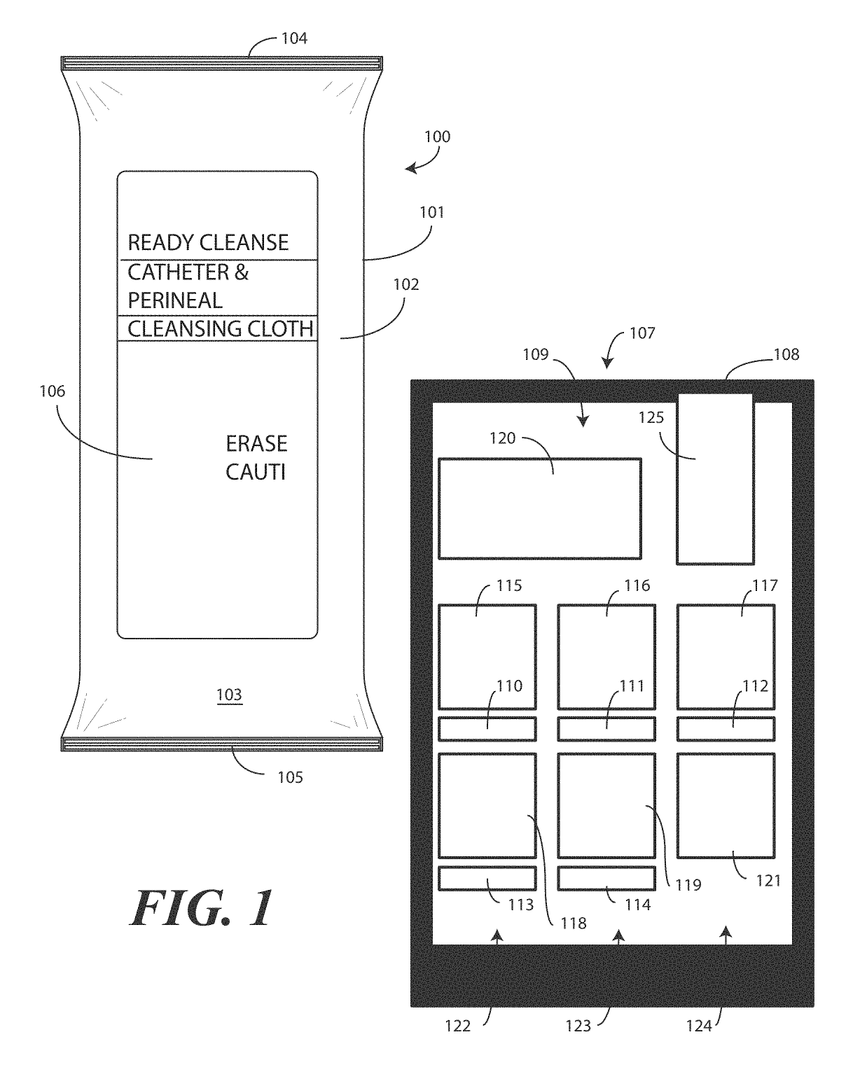

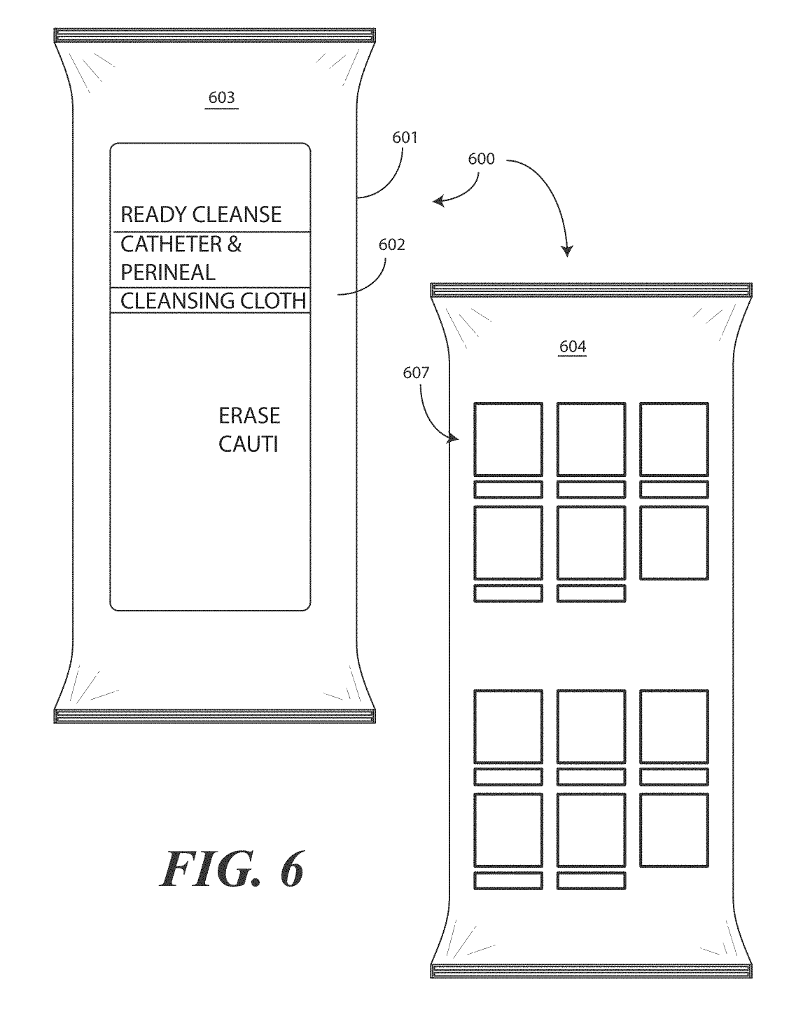

Turning now to FIG. 1, illustrated therein is one explanatory medical assembly 100 in accordance with one or more embodiments of the disclosure. In one embodiment, the medical assembly 100 includes a package 101 comprising an outer surface 102. In one embodiment, the outer surface 102 defines a major face 103 forming the top of the package 101. A second major face (one embodiment of which is shown in FIG. 6) defines the bottom of the package 101, while one or more minor faces define the sides of the package.

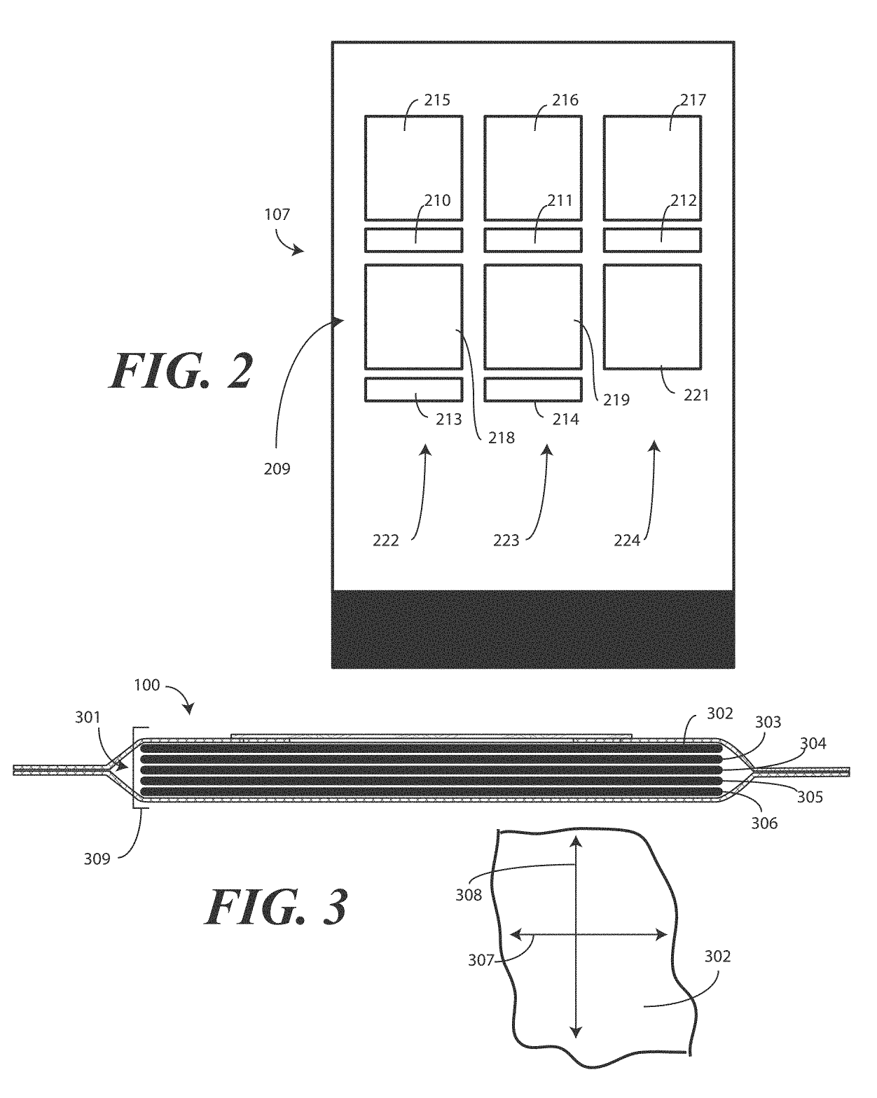

The package 101 can have one or more ends 104,105 that are sealed to define an interior compartment, which will be shown in more detail below with reference to FIG. 3. In one embodiment, the ends 104,105 are thermally sealed together. Other closure techniques can be used, including fusing, crimping, or bonding.

In one embodiment, the package 101 can be manufactured from a flexible film that is thermoplastic-based, foil based, or is another type of flexible material. In one or more embodiments, the package 101 is manufactured from a material that is not permeable to moisture. As will be explained in more detail below, in one or more embodiments, the package 101 can be used to enclose a stacked configuration of non-dispersible sheets.

The non-dispersible sheets can be pre-moistened in some use cases. A non-permeable material, such as polyester, polypropylene, polycarbonate, polyvinylidene fluoride, cellophane, polymethyl metacrylate, polystyrene, ethylene acrylic acid, polyvinyl chloride, acetate fiber, single or multi laminated films, or a combination thereof, can be incorporated into the package 101 prevent the loss of moisture from the pre-moistened sheets. Other suitable materials include metallic foils, synthetic fiber materials, coated materials, lined materials, and so forth.

The package 101 can be manufactured from one or more layers as well. In still other embodiments, the package 101 can be manufactured from a rigid material, such as a thermoplastic or metal as well. Other packaging configurations will be obvious to those of ordinary skill in the art having the benefit of this disclosure.

In one embodiment, the package 101 is substantially rectangular when viewed in plan view as shown in FIG. 1. However, it should be noted that the package 101 may be any shape, including square, oval, circular, free-form shapes, or any other desired shape.

In one embodiment, a closure is attached to the outer surface 102 of the package 101. In this embodiment, since the package 101 is manufactured from a flexible material, the closure is in the form of an aperture-sealing label 106 that is attached to the outer surface 102 along the major face 103 of the package 101. Had the package 101 been manufactured from a rigid material, the closure could have been a lid or other closure. Additionally, while the centrally disposed location along the major face 103 of the outer surface 102 of the package 101 is one possible location for the aperture-sealing label 106, the aperture-sealing label 106 can be disposed at other locations as well.

In one or more embodiments, printed instructions 107 can be included with the package 101 to form a medical assembly. In one embodiment, for example, the package 101 and printed instructions 107 are enclosed within an outer packaging. In one embodiment, the printed instructions 107 can include instructions telling a health care services provider, for example, how to clean a catheter insertion site, when to use each sheet disposed within the package 101, and when to discard each sheet taken from the package 101 to best reduce the chance for causing a catheter associated urinary tract infection. The printed instructions 107 can include pictures or illustrations showing visually how the various steps should be performed as well. Further the printed instructions 107 can notify the medical services provider that the sheets disposed within the package 101 are ordered corresponding to use during the catheter cleaning procedure prescribed by the printed instructions 107.

The printed instructions 107 can be configured in a variety of ways. Illustrating by example, in one embodiment the printed instructions 107 are disposed along a substantially flat substrate 108, such as a piece of paper, poster board, cardboard, card stock, film, laminate, plastic sheeting, or other substantially flat substrate upon which printing can be disposed. In one or more embodiments where the substantially flat substrate 108 is paper-based, the printed instructions 107 can be laminated as well.

While the substantially flat substrate 108 is singular in the embodiment of FIG. 1, with all information corresponding to properly cleaning a Foley catheter insertion site of a female disposed along a front major face (shown in FIG. 1), and information corresponding to properly cleaning a Foley catheter insertion site of a male disposed along a rear major face (shown in FIG. 2), the printed instructions 107 could be configured in other ways as well. For example, the printed instructions 107 could be configured as a stack of cards, a stack of flash cards, a bi-fold, tri-fold, or multi-fold pamphlet, scroll, or booklet. As will be shown in more detail below with reference to FIG. 6, the printed instructions 107 could also be disposed directly on the package 101 so as to be wholly functionally related to the substrate upon which they are disposed.

The front side of the printed instructions shown in FIG. 1 provides illustrative instructional material 109 suitable for use in cleaning Foley catheter insertion sites. In one embodiment, the instructional material 109 comprises text only. For example, in one embodiment the instructional material 109 comprises a description 120 of the contents of the package 101, and a series of method steps 110,111,112,113,114. In one embodiment, each of these method steps 110,111,112,113,114 includes an instruction to discard a sheet after executing the steps 110,111,112,113,114. Said differently, in one embodiment each step 110,111,112,113,114 includes an instruction to discard a sheet on a one-to-one basis with the step. Thus, step 110 would include an instruction to discard a sheet, while step 111 included another instruction to discard a sheet, and so forth.

In one or more embodiments, the printed instructions 107 further include a command 121, which can be configured as written or pictorial indicia, not to flush the non-dispersible sheets down a toilet. In one embodiment, the command 121 is “DO NOT FLUSH” written in large, bold letters along the printed instructions 107. The inclusion of the command 121 ensures that spent sheets are not disposed by flushing, but are instead disposed in a contained and hygienic fashion.

While text is one way to present instructional material 109 on the printed instructions, in other embodiments one or more pictorial images 115,116,117,118,119 can be included with the text to make the instructional material 109 more easily understandable. As they say, a pictorial image 115,116,117,118,119 can be worth a thousand words. Accordingly, including one or more pictorial images 115,116,117,118,119 can reduce the amount of text needed to convey the same message. In one embodiment, a picture 125 of the package 101 can be included to alert the medical services provider that the printed instructions 107 are to be used with the medical assembly 100.

In this illustrative embodiment, the instructional material 109 is arranged in three columnar sections 122,123,124, each including a method step configured as a pictorial image 115,116,117,118,119 and a corresponding method step 111,112,113,114. For example, a first step in a method is taught by pictorial image 115 and corresponding textual step 110. A second step in a method is taught by pictorial image 116 and corresponding textual step 111. A third step in a method is taught by pictorial image 117 and corresponding textual step 112. A fourth step in a method is taught by pictorial image 118 and corresponding textual step 113. A fifth step in a method is taught by pictorial image 119 and corresponding textual step 114. In one embodiment, five sheets are disposed within the package 101. Accordingly, in one embodiment each step of the method set forth by the pictorial images 115,116,117,118,119 and corresponding steps 111,112,113,114 correspond to a sheet on a one-to-one basis.

In one embodiment, one side of the printed instructions 107 discloses a method of cleaning a catheter insertion site when the catheter is inserted into a female patient, while another side of the printed instructions 107 discloses a method of cleaning a catheter insertion site when the catheter is inserted into a male patient. For example, the front side of the printed instructions 107, shown in FIG. 1, may disclose a method of cleaning a catheter insertion site when the catheter is inserted into a female patient, while the back side, shown in FIG. 2, can disclose a method of cleaning a catheter insertion site when the catheter is inserted into a male patient.

Turning now to FIG. 2, like the front side, the rear side can include instructional material 209 suitable for use in cleaning Foley catheter insertion sites where the catheter is inserted into a male patient. As with the front side, in one embodiment, the instructional material 209 comprises text only. For example, in one embodiment the instructional material 209 comprises a series of method steps 210,211,212,213,214. In one embodiment, each of these method steps 210,211,212,213,214 includes an instruction to discard a sheet after executing the steps 210,211,212,213,214. In other embodiments one or more pictorial images 215,216,217,218,219 can be included with the text to make the instructional material 209 more easily understandable.

In one or more embodiments, the printed instructions 107 further include a command 221, which can be configured as written or pictorial indicia, not to flush the non-dispersible sheets down a toilet. In one embodiment, the command 221 is “DO NOT FLUSH” written in large, bold letters along the printed instructions 107.

In this illustrative embodiment, the instructional material 209 is arranged in three columnar sections 222,223,225, each including a method step configured as a pictorial image 215,216,217,218,219 and a corresponding method step 211,212,213,214. For example, a first step in a method is taught by pictorial image 215 and corresponding textual step 210. A second step in a method is taught by pictorial image 216 and corresponding textual step 211. A third step in a method is taught by pictorial image 217 and corresponding textual step 212. A fourth step in a method is taught by pictorial image 218 and corresponding textual step 213. A fifth step in a method is taught by pictorial image 219 and corresponding textual step 214. In one embodiment, five sheets are disposed within the package (101). Accordingly, in one embodiment each step of the method set forth by the pictorial images 215,216,217,218,219 and corresponding steps 211,212,213,214 correspond to a sheet on a one-to-one basis.

Turning now to FIG. 3, a sectional view of the medical assembly 100 is shown. In one embodiment, a plurality of sheets 301 arranged in a stacked configuration 309. In one embodiment, each sheet is a non-dispersible sheet manufactured from needlepunched or spunlace material. It should be noted that each sheet 302,303,304,305,306 could be of any of a number of colors. Additionally, each sheet 302,303,304,305,306 could have visible indicia disposed thereon.

In one embodiment, the plurality of sheets 301 comprises a predefined number of sheets. In this illustrative embodiment, the predefined number of sheets is five sheets, as this corresponds to the five steps described above for cleaning a catheter insertion site.

The plurality of sheets 301 can be manufactured from an organic, inorganic, or blended organic/inorganic material. For example, in one embodiment, the each sheet 302,303,304,305,306 is a non-dispersible sheet manufactured from a needle-punched material. Examples of non-dispersible sheets include needlepunched and spunlace sheets. Examples of such sheets are marketed by Medline Industries under the names ReadyBath.sup.™ and AloeTouch.sup.™. These non-dispersible sheets can be configured as soft, single patient use, spunlace or needlepunched wipes that are quite gentle on the skin. Such non-dispersible sheets are versatile and convenient for use as wipes for everyday cleaning and incontinence care.

In one or more embodiments, the non-dispersible sheets are pre-moistened with rinse-free formula that cleans, moisturizes and soothes the skin. In one or more embodiments, the non-dispersible sheets can be pH-balanced for patient use, can be hypoallergenic, and alcohol free. In one or more embodiments, the non-dispersible sheets can be provided with a light, gender-neutral scent or, alternatively, free of fragrance.

In other embodiments, each sheet 302,303,304,305,306 can be manufactured from other materials. In some embodiments, for example, the plurality of sheets 301 can be manufactured from cloth or synthetic material. The plurality of sheets 301 can be dry, or alternatively pre-moistened with water or other solutions. Examples of moistening solutions include moisturizers, cleaning solutions, deodorizers, solvents, disinfectants, medications, skin care products, insect repellants, fragrances, and so forth. In one or more embodiments, the plurality of sheets 301 is manufactured from a material that is strong enough to prevent ripping or tearing of each sheet 302,303,304,305,306 during normal anticipated use.

It should be noted that the plurality of sheets 301 can take any of a variety of shapes and sizes. In one embodiment, each sheet 302,303,304,305,306 measures about eight inches square. The term “about” is intended to describe a dimension inclusive of manufacturing tolerances. Accordingly, a dimension of “about 8.11 inches” having a manufacturing tolerance of plus or minus 0.10 inches can be between 8.01 inches and 8.21 inches, inclusive.

In one embodiment, each sheet 302,303,304,305,306 is a non-dispersible sheet having a width 307 of between five and eight inches. In one embodiment, the non-dispersible sheets have a length 308 of between seven and twelve inches. Examples of sizes of the non-dispersible sheets include 5.5″×7.25″, 8″×8″, and 8″×12″. These dimension examples are illustrative only, as others will be obvious to those of ordinary skill in the art having the benefit of this disclosure.

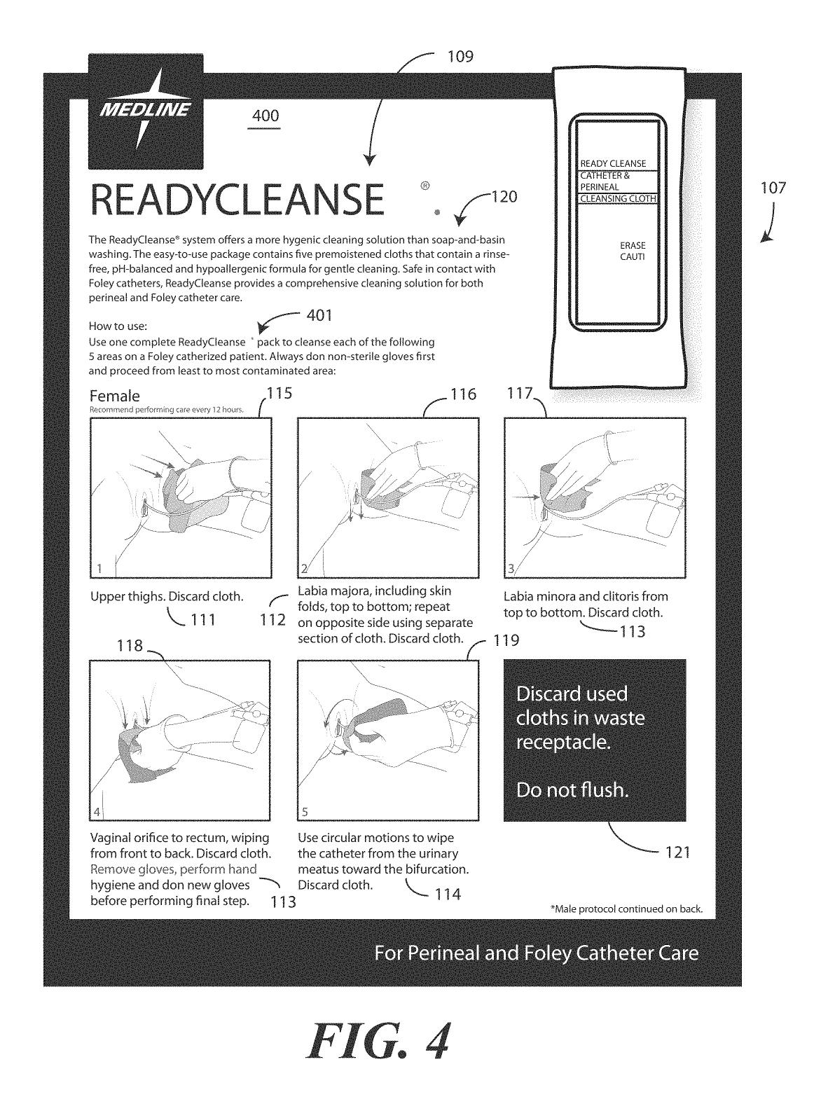

Turning now to FIG. 4, illustrated therein is one example of a front side 400 of printed instructions 107 configured in accordance with one or more embodiments of the disclosure. In one embodiment, the front side 400 of the printed instructions 107 illustrative instructional material 109 suitable for use in cleaning a Foley catheter insertion site where the catheter is inserted into a female patient.

In one embodiment, the instructional material 109 comprises a description 120 of the contents of the package 101. In one embodiment, this description 120 states that the medical assembly (100) offers a more hygienic cleaning solution that would a soap and basis washing approach. In one embodiment, the description 120 identifies the medical assembly (100) as an easy to use package containing five pre-moistened cloths that contain a rinse-free, pH-balanced, and hypoallergenic formula for gentle cleaning. In one embodiment, the description 120 states that the materials disposed on the sheets is safe when in contact with Foley catheters, and that the medical assembly (100) provides a comprehensive cleaning solution for both perineal and Foley catheter care.

In one embodiment, the instructional material 109 includes a summary 401 of how to use the medical assembly (100). For example, in one embodiment the summary 401 sates, “Use one complete ReadyCleans.sup.™ pack to cleanse each of the following 5 areas on a Foley catheterized patient.” The summary 401 may also include instructional details, such as “Always don non-sterile gloves first and proceed from the least to most contaminated area.”

In one embodiment, a method for cleaning a Foley catheter insertion site, where the catheter is inserted into a female, and using the medical assembly (100), is then described using one or more pictorial images 115,116,117,118,119 that are presented with corresponding method steps 110,111,112,113,114. In this illustrative embodiment, each cleaning operation set forth in the method is arranged as a pictorial image 115,116,117,118,119 and a corresponding method step 110,111,112,113,114. These pictorial images 115,116,117,118,119 and corresponding method steps 110,111,112,113,114 can be numbered to ensure that they are performed in the proper order. In one embodiment, each sheet of the medical assembly (100) is correspondingly numbered so that medical personnel can easily identify which sheet to use with which step.

For example, a first step in a method is taught by pictorial image 115 and corresponding textual step 110. In one embodiment, pictorial image 115 illustrates a female patient with a Foley catheter inserted into her urinary tract, with a medical professional using one sheet from the medical assembly (100) to wipe the patient's thighs. Corresponding method step 111 might state, “upper thighs,” thereby indicating that the first sheet of the medical assembly (100) should be used to clean the patient's thighs. In one embodiment, method step 111 further includes an instruction to discard the sheet after cleaning the patient's thighs. In one embodiment, this instruction states, “Discard cloth.”

A second step in a method is taught by pictorial image 116 and corresponding textual step 111. In one embodiment, pictorial image 116 illustrates a female patient with a Foley catheter inserted into her urinary tract, with a medical professional using one sheet from the medical assembly (100) to wipe the patient's labia majora, including skin folds, from top to bottom, on each side of the vagina. Corresponding method step 111 might state, “labia majora, including skin folds, top to bottom; repeat on opposite side using separate section of cloth,” thereby indicating that the second sheet of the medical assembly (100) should be used to clean the patient's private parts. In one embodiment, method step 111 further includes an instruction to discard the sheet after cleaning the patient's private parts. In one embodiment, this instruction states, “Discard cloth.”

A third step in a method is taught by pictorial image 117 and corresponding textual step 112. In one embodiment, pictorial image 117 illustrates a female patient with a Foley catheter inserted into her urinary tract, with a medical professional using one sheet from the medical assembly (100) to wipe the patient's labia minora, including clitoris, from top to bottom. Corresponding method step 112 might state, “labia minora, including clitoris, top to bottom,” thereby indicating that the third sheet of the medical assembly (100) should be used to clean more of the patient's private parts. In one embodiment, method step 112 further includes an instruction to discard the sheet after cleaning more of the patient's private parts. In one embodiment, this instruction states, “Discard cloth.”

A fourth step in a method is taught by pictorial image 118 and corresponding textual step 113. In one embodiment, pictorial image 118 illustrates a female patient with a Foley catheter inserted into her urinary tract, with a medical professional using one sheet from the medical assembly (100) to wipe the patient's vaginal orifice, to the rectum, from front to back. Corresponding method step 113 might state, “vaginal orifice to rectum, wiping from front to back,” thereby indicating that the fourth sheet of the medical assembly (100) should be used to clean even more of the patient's private parts. In one embodiment, method step 113 further includes an instruction to discard the sheet after cleaning the patient's private parts. In one embodiment, this instruction states, “Discard cloth.”

In one embodiment, textual step 113 further includes an intermediate step in the method. In one embodiment, this intermediate step recites, “Remove gloves, perform hand hygiene, and don new gloves before performing final step,” i.e., before performing the step set forth by pictorial image 119 and corresponding textual step 114.

A fifth step in a method is then taught by pictorial image 119 and corresponding textual step 114. In one embodiment, pictorial image 119 illustrates a female patient with a Foley catheter inserted into her urinary tract, with a medical professional using one sheet from the medical assembly (100) to wipe the catheter from the urinary meatus toward the bifurcation. Corresponding method step 114 might state, “Use circular motions to wipe the catheter from the urinary meatus toward the bifurcation,” thereby indicating that the final sheet of the medical assembly (100) should be used to clean the catheter. In one embodiment, method step 114 further includes an instruction to discard the sheet after cleaning the patient's private parts. In one embodiment, this instruction states, “Discard cloth.”

In one embodiment, five sheets (302,303,304,305,306) are disposed within the package (101) of the medical assembly (100). Accordingly, in one embodiment each step of the method set forth by the pictorial images 115,116,117,118,119 and corresponding steps 111,112,113,114 correspond to a sheet on a one-to-one basis. In one embodiment, to ensure that the discarded sheets are not flushed, a command 121 is present on the instructional material 109. In his illustrative embodiment, the command 121 states, “Discard used cloths in waste receptacle. DO NOT FLUSH.” This helps to ensure that non-dispersible sheets are not inadvertently flushed down the toilet.

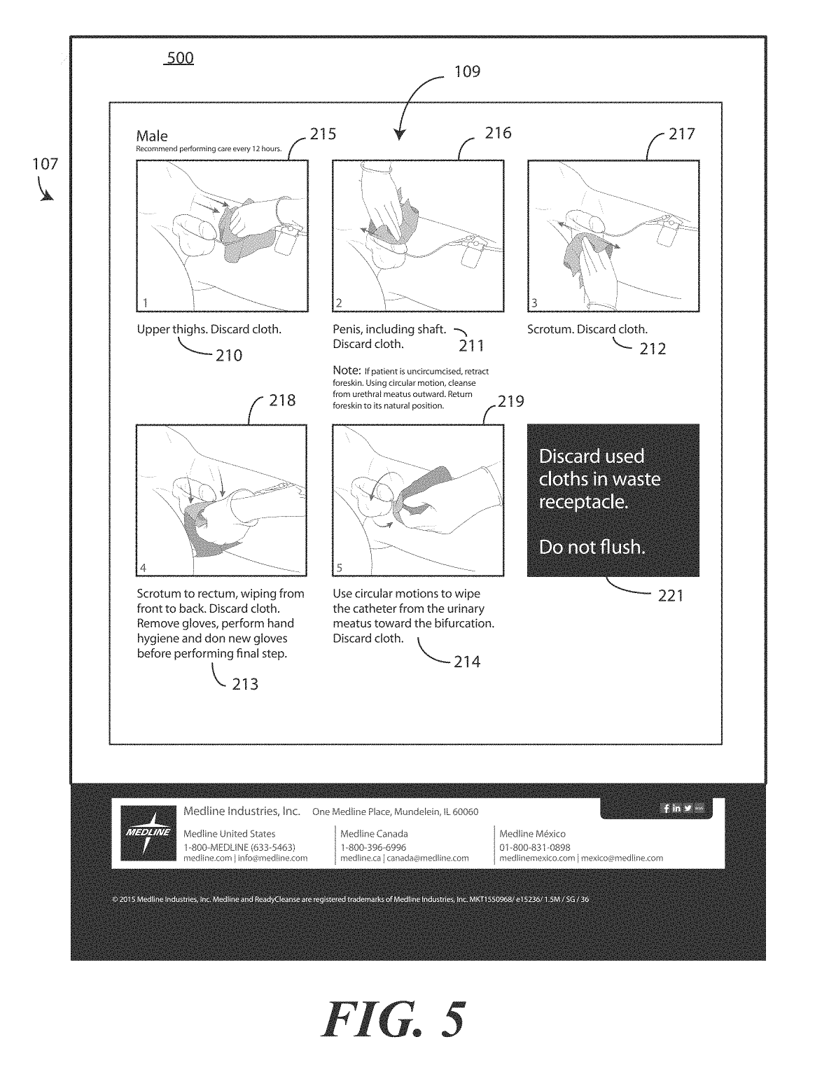

Turning now to FIG. 5, illustrated therein is one example of a rear side 500 of printed instructions 107 configured in accordance with one or more embodiments of the disclosure. In one embodiment, the front side 400 of the printed instructions 107 illustrative instructional material 109 suitable for use in cleaning a Foley catheter insertion site where the catheter is inserted into a female patient.

In one embodiment, the instructional material 209 comprises a method for cleaning a Foley catheter insertion site, where the catheter is inserted into a male, and using the medical assembly (100), described using one or more pictorial images 215,216,217,218,219 that are presented with corresponding method steps 210,211,212,213,214. In this illustrative embodiment, each cleaning operation set forth in the method is arranged as a pictorial image 215,216,217,218,219 and a corresponding method step 210,211,212,213,214. These pictorial images 215,216,217,218,219 and corresponding method steps 210,211,212,213,214 can be numbered to ensure that they are performed in the proper order. In one embodiment, each sheet of the medical assembly (100) is correspondingly numbered so that medical personnel can easily identify which sheet to use with which step.

For example, a first step in a method is taught by pictorial image 215 and corresponding textual step 210. In one embodiment, pictorial image 215 illustrates a male patient with a Foley catheter inserted into his urinary tract, with a medical professional using one sheet from the medical assembly (100) to wipe the patient's thighs. Corresponding method step 211 might state, “upper thighs,” thereby indicating that the first sheet of the medical assembly (100) should be used to clean the patient's thighs. In one embodiment, method step 211 further includes an instruction to discard the sheet after cleaning the patient's thighs. In one embodiment, this instruction states, “Discard cloth.”

A second step in a method is taught by pictorial image 216 and corresponding textual step 211. In one embodiment, pictorial image 216 illustrates a male patient with a Foley catheter inserted into his urinary tract, with a medical professional using one sheet from the medical assembly (100) to wipe the patient's penis, including the shaft. Corresponding method step 211 might state, “penis, including shaft,” thereby indicating that the second sheet of the medical assembly (100) should be used to clean the patient's private parts. In one embodiment, method step 211 further includes an instruction to discard the sheet after cleaning the patient's private parts. In one embodiment, this instruction states, “Discard cloth.”

A third step in a method is taught by pictorial image 217 and corresponding textual step 212. In one embodiment, pictorial image 217 illustrates a male patient with a Foley catheter inserted into his urinary tract, with a medical professional using one sheet from the medical assembly (100) to wipe the patient's scrotum. Corresponding method step 212 might state, “scrotum,” thereby indicating that the third sheet of the medical assembly (100) should be used to clean more of the patient's private parts. In one embodiment, method step 212 further includes an instruction to discard the sheet after cleaning more of the patient's private parts. In one embodiment, this instruction states, “Discard cloth.”

A fourth step in a method is taught by pictorial image 218 and corresponding textual step 213. In one embodiment, pictorial image 218 illustrates a male patient with a Foley catheter inserted into his urinary tract, with a medical professional using one sheet from the medical assembly (100) to wipe the patient's area between scrotum and rectum. Corresponding method step 113 might state, “scrotum to rectum, wiping from front to back,” thereby indicating that the fourth sheet of the medical assembly (100) should be used to clean even more of the patient's private parts. In one embodiment, method step 213 further includes an instruction to discard the sheet after cleaning the patient's private parts. In one embodiment, this instruction states, “Discard cloth.”

In one embodiment, textual step 213 further includes an intermediate step in the method. In one embodiment, this intermediate step recites, “Remove gloves, perform hand hygiene, and don new gloves before performing final step,” i.e., before performing the step set forth by pictorial image 219 and corresponding textual step 214.

A fifth step in a method is then taught by pictorial image 219 and corresponding textual step 214. In one embodiment, pictorial image 219 illustrates a male patient with a Foley catheter inserted into his urinary tract, with a medical professional using one sheet from the medical assembly (100) to wipe the catheter from the urinary meatus toward the bifurcation. Corresponding method step 214 might state, “Use circular motions to wipe the catheter from the urinary meatus toward the bifurcation,” thereby indicating that the final sheet of the medical assembly (100) should be used to clean the catheter. In one embodiment, method step 214 further includes an instruction to discard the sheet after cleaning the patient's private parts. In one embodiment, this instruction states, “Discard cloth.”

In one embodiment, five sheets (302,303,304,305,306) are disposed within the package (101) of the medical assembly (100). Accordingly, in one embodiment each step of the method set forth by the pictorial images 215,216,217,218,219 and corresponding steps 211,212,213,214 correspond to a sheet on a one-to-one basis. In one embodiment, to ensure that the discarded sheets are not flushed, a command 221 is present on the instructional material 209. In his illustrative embodiment, the command 221 states, “Discard used cloths in waste receptacle. DO NOT FLUSH.” This helps to ensure that non-dispersible sheets are not inadvertently flushed down the toilet.

Turning now to FIG. 6, illustrated therein is an alternate medical assembly 600 in accordance with one or more embodiments of the disclosure. As with the embodiment of FIG. 1, in this embodiment the medical assembly 600 includes a package 601 comprising an outer surface 602. In one embodiment, the outer surface 602 defines a major face 603 forming the top of the package 601. A second major face 604 defines the rear side of the package 601, while one or more minor faces define the sides of the package. The package 601 can be manufactured from a flexible film that is thermoplastic-based, foil based, or is another type of flexible material as previously described. The package 601 can also be used to enclose a stacked configuration of non-dispersible sheets as previously described.

In one or more embodiments, printed instructions 607 can be disposed along the second major face 604 of the medical assembly 600. In contrast to the printed instructions (107) of FIG. 1, which were enclosed within an outer packaging, in FIG. 6 the printed instructions 607 are printed directly on the package 601 so as to be functionally related to the substrate upon which they are disposed.

In one embodiment, the printed instructions 607 can include instructions telling a health care services provider, for example, how to clean a catheter insertion site, when to use each sheet disposed within the package 601, and when to discard each sheet taken from the package 601 to best reduce the chance for causing a catheter associated urinary tract infection. The printed instructions 607 can include pictures or illustrations showing visually how the various steps should be performed as well. Further the printed instructions 607 can notify the medical services provider that the sheets disposed within the package 601 are ordered corresponding to use during the catheter cleaning procedure prescribed by the printed instructions 607.

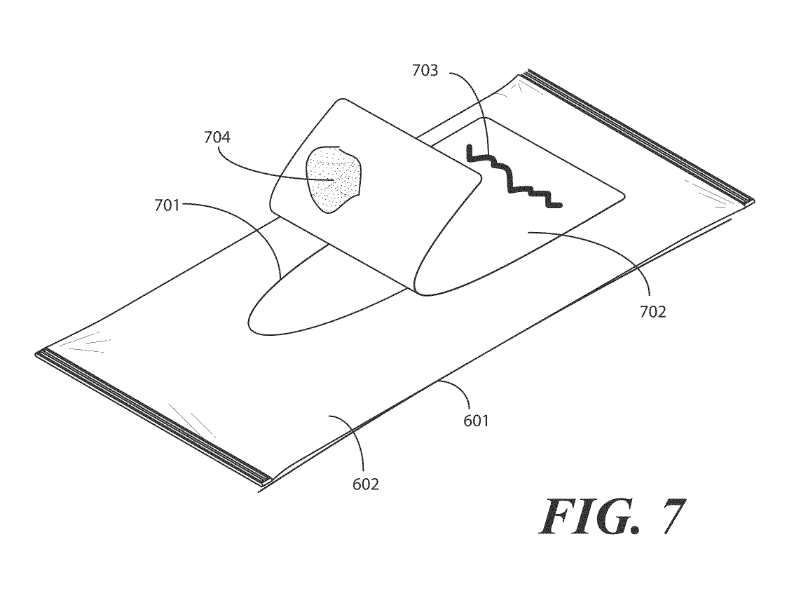

Turning now to FIG. 7, regardless of whether the package 601 of FIG. 6 or the package (107) of FIG. 1 is used, in one embodiment the outer surface 602 of the package 601 defines an aperture 701. The aperture 701 provides an opening to the interior compartment. As was described above with reference to FIG. 3, in one embodiment five sheets are arranged in a stacked configuration and are disposed within the interior compartment. In such a configuration, the sheets can be drawn from the package 601 through the aperture 701.

In one embodiment, the aperture 701 is cut into the package 601. In other embodiments, the package 601 can be scored or perforated, thereby requiring a user to tear the package along the score or perforation line to open the aperture 701. It should be noted that the aperture may be any desired shape and/or size, including the ovular shape shown in FIG. 7. In one or more embodiments, the aperture 701 is defined by abutting or overlapping edges of the package 601 that are not attached together.

In one embodiment, the aperture-sealing label 702 is attached to the outer surface 602 of the package 601 so as to cover and close the aperture 701. As shown in FIG. 7, the aperture-sealing label 702 is attached to the major face 603 of the outer surface 602 of the package 601 so as to cover, conceal, and close the aperture 701. However, as shown in FIG. 7, when the aperture-sealing label 702 is peeled back, the aperture 701 is exposed.

In one embodiment, the aperture-sealing label 702 comprises printed indicia 703 disposed thereon. The printed indicia 703 can include branding or other information identifying that a plurality of sheets are disposed within the package 601. In one embodiment, the printed indicia 703 include instructions for using the contents of the package 601. Other information suitable for inclusion within the printed indicia 703 will be obvious to those of ordinary skill in the art having the benefit of this disclosure.

In one or more embodiments, the aperture-sealing label 702 is selectively attachable to, and peelable from, the outer surface 602 of the package 601. In one embodiment, the aperture-sealing label 702 is attached to the package 601 such that it can be lifted and resealed repeatedly without damaging the package 601. For example, in one embodiment, the aperture-sealing label 702 has a releasable adhesive 704 disposed on the bottom side of the aperture-sealing label 702 so that it can selectively attach to, or be peeled from, the outer surface 602 of the package 601. In one or more embodiments, the aperture-sealing label 702 is to an aperture-sealing label 702 to selectively attach to the outer surface 602 to close the aperture 701, and to be selectively peelable from the outer surface 602 to expose the aperture 701.

It should be noted that a different type of closure could be used where the container is manufactured from a different material. Whether the closure is an aperture-sealing label 702, a lid, a flip-top, a press-on snap-fit closure, or other type of closure, the closure functions to selectively open allow a user to remove contents from the package 601 through the aperture 701, and in one or more embodiments, be re-closable to cover and/or conceal the aperture 701. Thus, the closure serves as a resealing device to seal the aperture 701 in one or more embodiments.

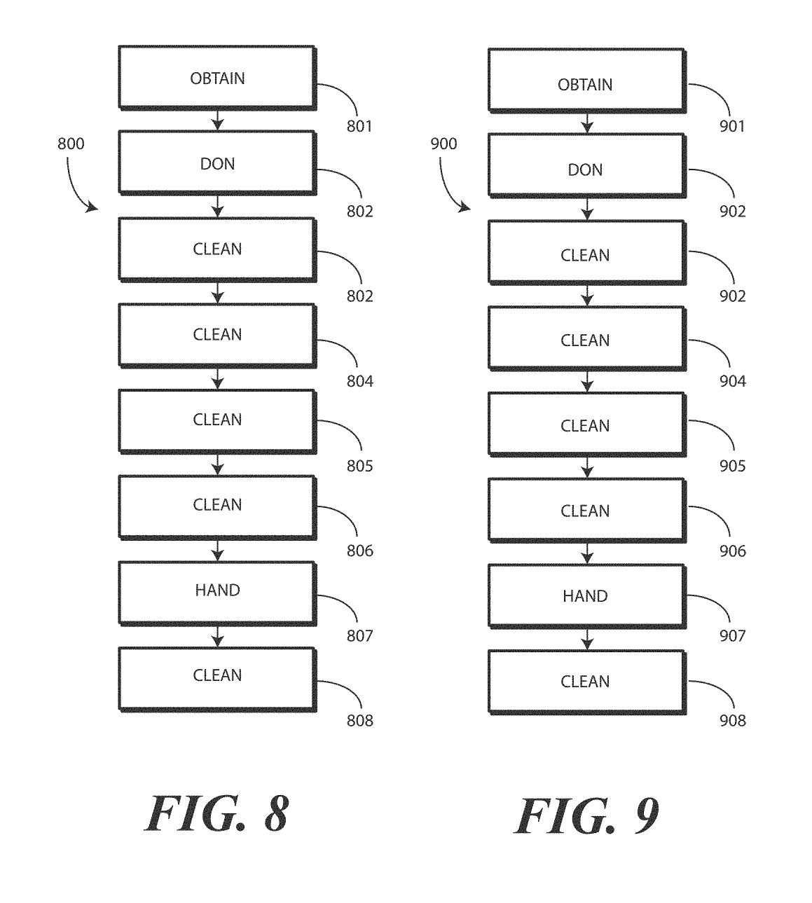

Turning now to FIG. 8, illustrated therein is one explanatory method 800 for cleaning a catheter insertion site where the catheter is inserted into a female patient. At step 801, one obtains a medical assembly comprising a package and a plurality of non-dispersible sheets. In one embodiment, the package contains five non-dispersible sheets. In one embodiment, the medical assembly includes printed instructions informing a health care services provider, for example, how to perform the method 800 of FIG. 8. In one embodiment, these instructions include how to clean a catheter insertion site, when to use each sheet disposed within the package, and when to discard each sheet taken from the package to best reduce the chance for causing a catheter associated urinary tract infection. The printed instructions can include pictures or illustrations showing visually how the various steps should be performed as well. Further the printed instructions can notify the medical services provider that the sheets disposed within the package are ordered corresponding to use during the catheter cleaning procedure prescribed by the printed instructions.

At step 802, the method 800 includes donning non-sterile gloves. In one embodiment, these non-sterile gloves are included with the medical assembly obtained at step 801.

At step 803, the method 800 includes drawing a first cloth from the medical assembly obtained at step 801 and cleaning a patient's upper thighs. In one embodiment, step 803 further includes discarding the first cloth after cleaning the patient's upper thighs. In one embodiment, the method includes discarding the first cloth by other than flushing the cloth down the toilet.

At step 804, the method 800 includes drawing a second cloth from the medical assembly obtained at step 801 and cleaning a patient's labia majora, including skin folds, top to bottom, and repeating on the opposite side of the vagina using a separate section of the second cloth. In one embodiment, step 804 further includes discarding the second cloth after performing the cleaning occurring in step 804. In one embodiment, the method includes discarding the second cloth by other than flushing the cloth down the toilet.

At step 805, the method 800 includes drawing a third cloth from the medical assembly obtained at step 801 and cleaning a patient's labia minora and clitoris, from top to bottom. In one embodiment, step 805 further includes discarding the second cloth after performing the cleaning occurring in step 805. In one embodiment, the method includes discarding the third cloth by other than flushing the cloth down the toilet.

At step 806, the method 800 includes drawing a fourth cloth from the medical assembly obtained at step 801 and cleaning a patient's vaginal orifice to their rectum, wiping front to back. In one embodiment, step 806 further includes discarding the second cloth after performing the cleaning occurring in step 806. In one embodiment, the method includes discarding the fourth cloth by other than flushing the cloth down the toilet.

Optional step 807 comprises removing the non-sterile gloves donned at step 802, performing hand hygiene by washing hands or applying hand sanitizer, and donning new non-sterile gloves.

At step 808, the method 800 includes drawing a fifth cloth from the medical assembly obtained at step 801 and cleaning a patient's catheter, using a circular motion, from the urinary meatus toward the bifurcation of the catheter. In one embodiment, step 808 further includes discarding the fifth cloth after performing the cleaning occurring in step 808. In one embodiment, the method includes discarding the fifth cloth by other than flushing the cloth down the toilet.

Turning now to FIG. 9, illustrated therein is one explanatory method 900 for cleaning a catheter insertion site where the catheter is inserted into a male patient. At step 901, one obtains a medical assembly comprising a package and a plurality of non-dispersible sheets. In one embodiment, the package contains five non-dispersible sheets. In one embodiment, the medical assembly includes printed instructions informing a health care services provider, for example, how to perform the method 900 of FIG. 9. In one embodiment, these instructions include how to clean a catheter insertion site, when to use each sheet disposed within the package, and when to discard each sheet taken from the package to best reduce the chance for causing a catheter associated urinary tract infection. The printed instructions can include pictures or illustrations showing visually how the various steps should be performed as well. Further the printed instructions can notify the medical services provider that the sheets disposed within the package are ordered corresponding to use during the catheter cleaning procedure prescribed by the printed instructions.

At step 902, the method 900 includes donning non-sterile gloves. In one embodiment, these non-sterile gloves are included with the medical assembly obtained at step 901.

At step 903, the method 900 includes drawing a first cloth from the medical assembly obtained at step 901 and cleaning a patient's upper thighs. In one embodiment, step 903 further includes discarding the first cloth after cleaning the patient's upper thighs. In one embodiment, the method includes discarding the first cloth by other than flushing the cloth down the toilet.

At step 904, the method 900 includes drawing a second cloth from the medical assembly obtained at step 901 and cleaning a patient's penis, including the shaft. In one embodiment, step 904 further includes discarding the second cloth after performing the cleaning occurring in step 904. In one embodiment, the method includes discarding the second cloth by other than flushing the cloth down the toilet.

At step 905, the method 900 includes drawing a third cloth from the medical assembly obtained at step 901 and cleaning a patient's scrotum. In one embodiment, step 905 further includes discarding the second cloth after performing the cleaning occurring in step 905. In one embodiment, the method includes discarding the third cloth by other than flushing the cloth down the toilet.

At step 906, the method 900 includes drawing a fourth cloth from the medical assembly obtained at step 901 and cleaning a patient's scrotum to their rectum, wiping front to back. In one embodiment, step 906 further includes discarding the second cloth after performing the cleaning occurring in step 806. In one embodiment, the method includes discarding the fourth cloth by other than flushing the cloth down the toilet.

Optional step 907 comprises removing the non-sterile gloves donned at step 902, performing hand hygiene by washing hands or applying hand sanitizer, and donning new non-sterile gloves.

At step 908, the method 900 includes drawing a fifth cloth from the medical assembly obtained at step 901 and cleaning a patient's catheter, using a circular motion, from the urinary meatus toward the bifurcation of the catheter. In one embodiment, step 908 further includes discarding the fifth cloth after performing the cleaning occurring in step 908. In one embodiment, the method includes discarding the first cloth by other than flushing the cloth down the toilet.

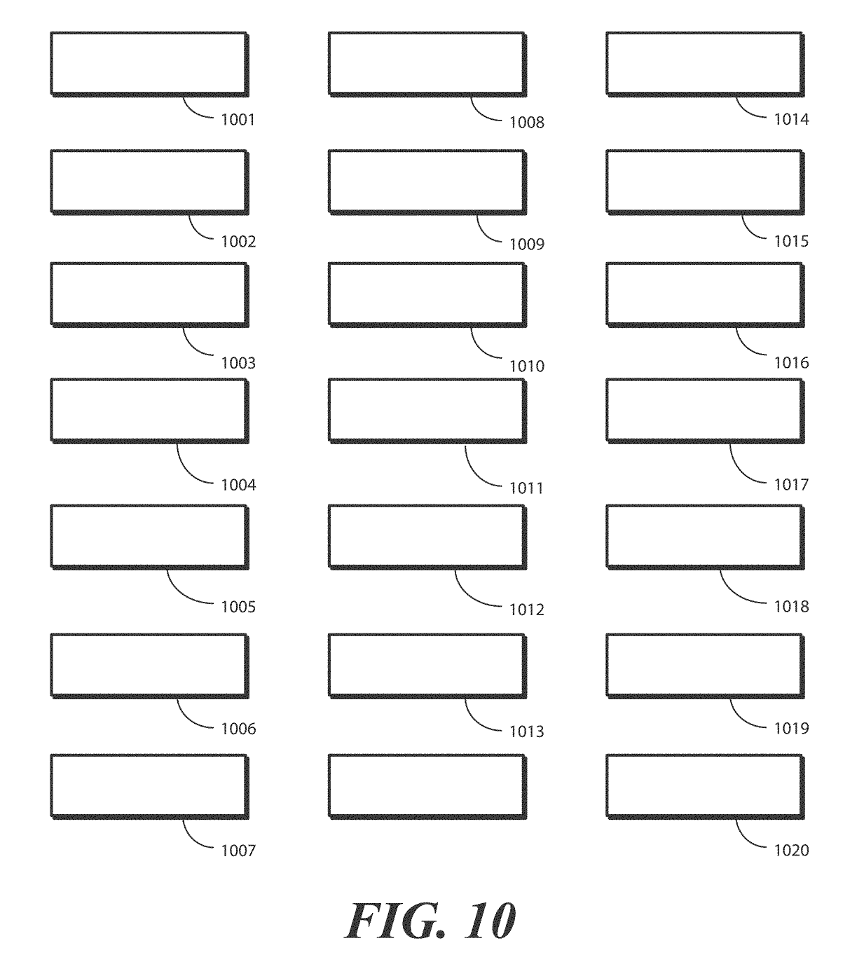

Turning now to FIG. 10, illustrated therein are various embodiments of the disclosure. At 1001, a medical assembly comprises a package comprising an outer surface having a major face defining an aperture. At 1001, the package comprises an aperture sealing label to selectively attach to the outer surface to close the aperture and to be selectively peelable from the outer surface to expose the aperture. At 1001, the medical assembly comprises five sheets arranged in a stacked configuration within the package. At 1001, the medical assembly comprises printed instructions defining five steps instructing when to use each sheet disposed within the package, and when to discard the each sheet disposed within the package, to clean a Foley catheter insertion site while reducing chances for causing a catheter associated urinary tract infection.

At 1002, the printed instructions of claim 1 comprise one or more of pictures or illustrations showing visually how to perform the five steps. At 1003, the printed instructions of 1002 further comprise instructions that the five sheets disposed within the package are ordered corresponding to use during a catheter cleaning procedure prescribed by the printed instructions.

At 1004, the printed instructions of 1001 are disposed along a substantially flat substrate. At 1005, all information corresponding to properly cleaning the Foley catheter insertion site of a female is disposed along a front major face of the substantially flat substrate of 1004, and all information corresponding to properly cleaning the Foley catheter insertion site of a male is disposed along a rear major face of the substantially flat substrate of 1004.

At 1006, the printed instructions of 1001 are disposed along the package. At 1007, the printed instructions comprising instructional material comprise a description of contents of the package and a series of method steps, wherein each step includes an instruction to discard a sheet after executing the each step on a one-to-one basis with the each step.

At 1008, the printed instructions of 1007 further comprise a command not to flush the each sheet down a toilet. At 1009, the command of 1008 is DO NOT FLUSH. At 1010, the printed instructions of 1001 further comprising a picture of the package indicating that the printed instructions are to be used with the medical assembly. At 1011, the printed instructions of 1001 comprise instructional material arranged in three columnar sections, with each columnar section including a method step configured as a pictorial image and a corresponding textual method step description.

At 1012, a method of cleaning a Foley catheter insertion site comprises obtaining a medical assembly comprising a package comprising an outer surface having a major face defining an aperture, five sheets arranged in a stacked configuration within the package, and printed instructions defining five steps instructing when to use each sheet disposed within the package, and when to discard the each sheet disposed within the package, to clean the Foley catheter insertion site while reducing chances for causing a catheter associated urinary tract infection. At 1012, the method includes drawing a first sheet from the package, cleaning a portion of a patient with the first sheet, and discarding the first sheet without flushing the first sheet. At 1012, the patient can be male or female.

At 1013-1016, the patient is female. At 1013, the method of 1012 further comprises drawing second sheet from the package, cleaning labia majora of the patient, including skin folds, top to bottom with a first portion of the second sheet, repeating the cleaning labia major of the patient on a second vaginal side, top to bottom, with a second portion of the second sheet that is different from the first portion, and discarding the second sheet without flushing the second sheet.

At 1014, the method of 1012 further comprises drawing a second sheet from the package, cleaning labia minora and a clitoris of the patient, from top to bottom, using the second sheet, and discarding the second sheet without flushing the second sheet. At 1015, the method of 1012 further comprises drawing a second sheet from the package, cleaning an area between a vaginal orifice of the patient and a rectum of the patient, wiping from the vaginal orifice to the rectum, with the second cloth, and discarding the second sheet without flushing the second sheet. At 1016, the method of 1012 further comprises drawing a second sheet from the package, cleaning, using a circular motion, a Foley catheter inserted into the Foley catheter insertion site from a urinary meatus of the patient to a bifurcation of the Foley catheter with the second sheet, and discarding the second sheet without flushing the second sheet.

At 1017-1020, the patient is male. At 1017, the method of 1012 further comprises drawing second sheet from the package, cleaning a penis of the patient, including a shaft of the penis, with the second sheet, and discarding the second sheet without flushing the second sheet. At 1018, the method of 1017 further comprises drawing a third sheet from the package, cleaning, using a circular motion, a Foley catheter inserted into the Foley catheter insertion site from a urinary meatus of the patient to a bifurcation of the Foley catheter with the third sheet, and discarding the third sheet without flushing the second sheet.

At 1019, the method of 1012 further comprises drawing a second sheet from the package cleaning a scrotum of the patient, using the second sheet, and discarding the second sheet without flushing the second sheet. At 1020, the method of 1012 further comprises drawing a second sheet from the package, cleaning an area between a scrotum of the patient and a rectum of the patient, wiping from the scrotum to the rectum, with the second cloth, and discarding the second sheet without flushing the second sheet. Embodiments other than those shown in FIG. 10, which are in accordance with one or more embodiments of the disclosure, will be obvious to those of ordinary skill in the art having the benefit of this disclosure.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims. Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims of this application and all equivalents thereof.

What is claimed is:

1. A medical assembly, comprising:
a package comprising an outer surface having a major face defining an aperture;
an aperture sealing label to selectively attach to the outer surface to close the aperture and to be selectively peelable from the outer surface to expose the aperture; and
five sheets arranged in a stacked configuration within the package; and
printed instructions defining five steps instructing when to use each sheet disposed within the package, and when to discard the each sheet disposed within the package, to clean a Foley catheter insertion site while reducing chances for causing a catheter associated urinary tract infection.

2. The medical assembly of claim 1, the printed instructions comprising one or more of pictures or illustrations showing visually how to perform the five steps.

3. The medical assembly of claim 2, the printed instructions further instructing that the five sheets disposed within the package are ordered corresponding to use during a catheter cleaning procedure prescribed by the printed instructions.

4. The medical assembly of claim 1, wherein the printed instructions are disposed along a substantially flat substrate.

5. The medical assembly of claim 4, wherein all information corresponding to properly cleaning the Foley catheter insertion site of a female is disposed along a front major face of the substantially flat substrate, and all information corresponding to properly cleaning the Foley catheter insertion site of a male is disposed along a rear major face of the substantially flat substrate.

6. The medical assembly of claim 1, wherein the printed instructions are disposed along the package.

7. The medical assembly of claim 1, the printed instructions comprising instructional material comprising:
a description of contents of the package; and
a series of method steps, wherein each step includes an instruction to discard a sheet after executing the each step on a one-to-one basis with the each step.

8. The medical assembly of claim 7, the printed instructions further comprising a command not to flush the each sheet down a toilet.

9. The medical assembly of claim 8, wherein the command is DO NOT FLUSH.

10. The medical assembly of claim 1, the printed instructions further comprising a picture of the package indicating that the printed instructions are to be used with the medical assembly 100.

11. The medical assembly of claim 1, the printed instructions comprising instructional material arranged in three columnar sections, each columnar section including a method step configured as a pictorial image and a corresponding textual method step description.

12. A method of cleaning a Foley catheter insertion site, the method comprising:
obtaining a medical assembly comprising:
a package comprising an outer surface having a major face defining an aperture;
five sheets arranged in a stacked configuration within the package; and
printed instructions defining five steps instructing when to use each sheet disposed within the package, and when to discard the each sheet disposed within the package, to clean the Foley catheter insertion site while reducing chances for causing a catheter associated urinary tract infection;
drawing a first sheet from the package;
cleaning a portion of a patient with the first sheet; and
discarding the first sheet without flushing the first sheet.

13. The method of claim 12, further comprising:
drawing a second sheet from the package;
cleaning labia majora of the patient, including skin folds, top to bottom with a first portion of the second sheet;
repeating the cleaning labia majora of the patient on a second vaginal side, top to bottom, with a second portion of the second sheet that is different from the first portion; and
discarding the second sheet without flushing the second sheet.

14. The method of claim 12, further comprising:
drawing a second sheet from the package;
cleaning labia minora and a clitoris of the patient, from top to bottom, using the second sheet; and
discarding the second sheet without flushing the second sheet.

15. The method of claim 12, further comprising:
drawing a second sheet from the package;
cleaning an area between a vaginal orifice of the patient and a rectum of the patient, wiping from the vaginal orifice to the rectum, with the second sheet; and
discarding the second sheet without flushing the second sheet.

16. The method of claim 12, further comprising:
drawing a second sheet from the package;
cleaning, using a circular motion, a Foley catheter inserted into the Foley catheter insertion site from a urinary meatus of the patient to a bifurcation of the Foley catheter with the second sheet; and
discarding the second sheet without flushing the second sheet.

17. The method of claim 12, further comprising:
drawing a second sheet from the package;
cleaning a penis of the patient, including a shaft of the penis, with the second sheet; and
discarding the second sheet without flushing the second sheet.

18. The method of claim 17, further comprising:
drawing a third sheet from the package;
cleaning, using a circular motion, a Foley catheter inserted into the Foley catheter insertion site from a urinary meatus of the patient to a bifurcation of the Foley catheter with the third sheet; and
discarding the third sheet without flushing the second sheet.

19. The method of claim 12, further comprising:
drawing a second sheet from the package;
cleaning a scrotum of the patient, using the second sheet; and
discarding the second sheet without flushing the second sheet.

20. The method of claim 12, further comprising:
drawing a second sheet from the package;
cleaning an area between a scrotum of the patient and a rectum of the patient, wiping from the scrotum to the rectum, with the second sheet; and
discarding the second sheet without flushing the second sheet.
